(19) United States
(12) Patent Application Publication
(10) Pub. No.: US 2010/0292526 A1
(43) Pub. Date: Nov. 18, 2010

(54) BRACHYTHERAPY APPLIANCE AND METHOD

(76) Inventor: Ethan J. Schuman, University City, MO (US)

Correspondence Address: MATTHEWS EDWARDS LLC, 514 EARTH CITY PLAZA, SUITE 131, EARTH CITY, MO, 63045, US

(21) Appl. No.: 12/812,851

(22) PCT Filed: Jan. 14, 2008
(86) PCT No.: PCT/US08/00472

§ 371 Date: Jul. 14, 2010

(51) Int. Cl. A61M 36/04; A61N 5/00; B23P 17/04

(52) U.S. Cl. 600/3; 29/592

(57) ABSTRACT

A brachytherapy appliance removably positionable on or against a surface of a living body, such as the body of a human or animal, for positioning a therapeutic delivery device in a desired precise location in relation to a selected portion of the body in or adjacent to the surface and containing a cancer, tumor, or other diseased tissue, for delivering a therapy, particularly radiation, to the diseased tissue, and a method of making the appliance so as to conform to the body surface, based on a computerized three dimensional model or map generated from a scan of the body region, the appliance being particularly adapted for use in a body cavity such as the oral cavity.

This application is submitted under 35 U.S.C. 371 claiming priority to PCT/US2008/00472, filed Jan. 14, 2008.

### TECHNICAL FIELD

This invention relates generally to an appliance removably positionable on or against a surface of a living body, such as the body of a human or animal, for positioning a therapeutic delivery device in a desired precise location in relation to a selected portion of the body in or adjacent to the surface and containing a cancer, tumor, or other diseased tissue, for delivering a therapy, particularly radiation, to the diseased tissue, and a method of making the appliance so as to conform to the body surface, based on a computerized three dimensional model or map generated from a scan of the body region, the appliance being particularly adapted for use in a body cavity such as the oral cavity.

### BACKGROUND ART

In the field of medicine, there are courses of treatment for diseases and injuries which are facilitated by placement of a therapeutic device within a body cavity, in a particular relation to a selected, or a predetermined, region thereof, or adjacent tissue, for delivering a therapeutic dosage. For instance, in oncology, particularly for the treatment of mouth and/or throat cancers, and more generally cancers of the head and/or throat, breast and prostate, and also non-cancerous diseases, such as non-malignant tumors and neuromas, therapeutic benefit has been achieved by brachytherapy, that is, the placement of radioactive elements, also referred to as radioactive seeds, in proximity to, or surgically directly in the cancer or cancerous or other diseased tissue, for directing radiation thereat. However, a side effect that has been observed is relatively uncontrolled irradiation of the surrounding tissue and/or organs. Specifically in this regard in relation to cancers and other diseases of the neck, head and mouth, is injury to and loss of teeth, and injury to salivary and other glands such as those relating to the sense of taste and smell, which is undesirable for many reasons.

It is thus sought to provide an appliance configured and/or adapted for placement in conforming relation to a body surface, with particular utility for use in cooperation with a surface within a body cavity, such as, but not limited to, the oral cavity, which allows removably or temporarily positioning of one or more therapy delivery elements, particularly radiation emitting elements, such as radioactive seeds, wires or the like, in precise relation to a cancer or other diseased tissue in or adjacent to the surface, which provides shielding for other regions of healthy tissue, glands and/or organs, as well as teeth, from the radiation emitted by the radioactive element or elements. Further in this regard, it would be advantageous to provide an appliance custom configured to conform to the shapes and contours of the body surface, so as to be positionable and automatically retained thereagainst in conforming relation thereto in a selected precise location and orientation in relation to the tissue which is to be irradiated, while providing radiation shielding for the other tissue and organs not intended to be irradiated. In such appliance, it would be advantageous to provide at least one receptacle or passage into which a therapeutic device, such as a seed or seeds, or a wire having a radioactive tip or other region, or other radioactive medium, is removably insertable, so as to be positioned for delivery of a radioactive dose in precise relation to a cancer to be irradiated for a specific time or specific therapeutic dosage.

### SUMMARY OF THE INVENTION

According to the invention, a brachytherapy appliance and method of practicing the same, which overcomes one or more of the problems and/or shortcomings, and provides one or more of the advantages set forth above, is disclosed.

According to a preferred aspect of the invention, a brachytherapy appliance is provided including a body portion fabricated to conform to contours of a body surface, which can be a surface within the oral cavity The appliance is positionable in close proximity to or against the body surface so as to be retained thereagainst or thereon, the body portion including at least one receptacle or passage disposed in or extending through a portion thereof adapted for receiving and holding a therapeutic element in precise relation to a cancer or cancerous tissue in the cavity or in nearby tissue, and which optionally includes shielding for protecting other tissue and body portions from the therapeutic element or effects thereof, particularly radiation therefrom. A therapeutic element is removably insertable in the body portion receptacle or passage in a predetermined position and/or orientation for delivering a therapeutic treatment, e.g., radiation, toward the cancer or cancerous tissue. Examples of a therapeutic element include, but are not limited to, a radiation delivery element such as a wire including a radiation emitting or irradiated portion, such as a tip or other segment, or one or more radiation emitting seeds, or other radioactive media. By providing the therapeutic, e.g. radioactive, media separately of the body portion, advantages include that the number and dosing, e.g., radioactivity, of the radioactive media can be varied, for instance, to allow changing the radiation dose or time of dosage over the course of therapy, or otherwise. This also allows use of the same radioactive media with more than one appliance, and also facilitates handling and storage of the radioactive media, and safe disposal of the non-radioactive body portion.

According to another preferred aspect of the invention, the body portion is configured or custom fit to fit securely on the upper teeth in the oral cavity, if any, or to the upper edentulous arch, such as by molding, so as to be positioned in the superior region of the oral cavity. The body portion is fabricated from a suitable polymeric material, such as, but not limited to, a suitable well known, commercially available moldable dental acrylic, such as, but not limited to, a methyl methacrylate. The at least one receptacle or passage will be molded into the body portion, and will comprise a tube or catheter adapted for receiving the radiation emitting media, such as a wire, therein, the tube or catheter being routed so as to have a portion or opening positioned to be disposed in desired relation to the cancer when the body portion is properly positioned in the body cavity, for delivery of the desired therapy, i.e., irradiation and destruction of the cancerous or diseased tissue or cells. The body portion additionally may include one or more layers of shielding material embedded therein, such as a lead shielding material, positioned for shielding adjacent healthy tissue, organs, teeth, or other vulnerable body parts, from radiation emitted by a radioactive region or portion of a wire or other radiation emitting media inserted into the tube or catheter. The lead shielding material, in turn, is incorporated into the body portion such that when the body portion is positioned in the oral cavity, the lead material will not contact the adjacent tissue, nor will the lead material be exposed to saliva or other fluids present in the cavity, which could carry contaminants to the surrounding tissue. It is also contemplated that an appliance of the invention can be configured for fitting in other regions of the oral cavity, such as on the lower teeth and/or edentulous arch, for treatment of cancers or diseases in the vicinity thereof.

According to still another preferred aspect of the invention, the therapeutic media comprises a wire removably insertable into the tube or catheter, and can be, for instance, a stainless steel wire, and can be, for instance, tipped with iridium 192, or another suitable radioactive substance. The wire is of a suitable length so as to extend through the tube or catheter so as to position the irradiated portion in the desired location in relation to the cancer or other diseased tissue, hereinafter sometimes referred to as “the cancer”, with the opposite end of the wire protruding from the appliance, and more preferably, also from the body cavity so as to be graspable for insertion and removal from the appliance. The wire can also include indicia along the length thereof for indicating proper placement of the tip or other irradiated portion at the desired location within the appliance, and thus in relation to the cancer to be irradiated.

According to a further preferred aspect of the invention, the body portion of the appliance will include multiple ones of the tubes or catheters, as required for holding the media necessary for treating, e.g., irradiating, the cancer to the desired extent. As an example, a plurality of the tubes or catheters will be arranged in a required array, such as, a linear, side-by-side array, for providing a desired pattern and/or dose of therapy, e.g., radiation, to the cancer. The required location of the tubes or catheters within the body portion of the appliance will be determined by the location of the cancer or diseased tissue, as found using common techniques, such as CT scanning or the like. The duration and/or dosage of treatment will be determined by an oncology professional or physicist.

According to a still further preferred aspect of the invention, a method of use of the appliance will include steps of molding the body portion to a surface region of a body, e.g., within a body cavity, such as the oral cavity, including installing the shielding, if used, and the at least one tube or catheter in the respective locations for holding the media for providing the therapeutic dosing to the cancer. The radioactive wire or wires or other media are then inserted into the one or more catheters or tubes, respectively, to position the therapeutic element or elements, e.g., radioactive seed or tip, in the desired relation to the cancer or diseased tissue. The appliance will then be left in place for the period of time necessary for delivery of the determined therapeutic dose to the cancer or diseased tissue. The wire or wires are then removed from the appliance, and the appliance removed from the subject body surface.

According to still another preferred aspect of the invention, a method of making the brachytherapy appliance involves utilizing a suitable three dimensional computer-generated model or rendering of a portion of a body against which, or adjacent to which, the appliance is to be located. The computer model can be generated using a suitable scanning device capable of modeling or mapping a body surface, such as, but not limited to, a CT scanning device such as a conventional multi-slice CT (MDCT) scanner, or a dental (cone-beam) CT scanner, or an MRI scanner. From this model, the topography (shape) of the surface of the body against which, or adjacent to which, the appliance is to be located, will be ascertained. This can include both soft (e.g., soft tissue) and hard (e.g., tooth) surfaces, and can be developed using known surface discernment and mapping techniques. A three dimensional physical model of this surface will then be produced. This three dimensional physical model can be of the body surface against which, or adjacent to which, the appliance is to be placed, and to which the appliance will be molded or conformed, or it can be the surface of the appliance that will be placed against, or adjacent to, the body surface, so as to essentially be a mirror image of, or mate to, that surface. The physical model can be produced, for instance, using a commercially available computer controlled lithography process, such as by solidifying liquid photopolymer into the three dimensional shape, milling a urethane or other suitable polymer block into the shape, or other three dimensional physical modeling technique used for making medical models.

If the physical model is of the body surface, the appliance can be made in any suitable manner, by molding or conforming the appliance to that surface, including in any of the above described ways, for instance by molding the appliance to the physical model, wherein the catheter or catheters are positioned on the model and are held in place using wax or adhesives. Also, the catheter or catheters can be set a required or desired distance from the surface of the physical model. Shielding can be applied as required or directed. Then, a vacuum form plastic shell can be sucked down on the model and catheter or catheters, and shielding, if present, to form the appliance. The formed plastic shell is then trimmed if required and the appliance is complete.

If the physical model is to be of the appliance itself, according to one preferred aspect of the invention, the initial conforming layer is produced, to provide a base for positioning the catheter or catheters, using any of the techniques discussed above, such as the lithographic technique. The catheter or catheters, and shielding if required, are then added to the appliance, and the thickness of the appliance can be increased, if required, using an appropriate method, such as using layers of plastics material. As an alternative, the body of the appliance can be produced, including one or more passages therein which will serve as the catheter or catheters, or for receiving the catheter or catheters, and the shielding, if required, added at an appropriate point in the fabrication process.

### BRIEF DESCRIPTION OF THE DRAWINGS

FIG. 1 is a top view of aspects of a brachytherapy appliance according to the present invention, including a plurality of catheters for receiving radioactive tipped wires, respectively, for dosing a cancer with radiation;

FIG. 2 is another top view of the brachytherapy appliance, illustrating alternative locations for the catheters;

FIG. 3 is a side view of the brachytherapy appliance;

FIG. 4 is a sectional view of the brachytherapy appliance, taken along line 4-4 of FIG. 3;

FIG. 4a is a sectional view of the appliance illustrating alternative shielding of the invention;

FIG. 4b is a top view of the appliance, illustrating another alternative shielding;

FIG. 4c is another top view of the appliance, illustrating another alternative shielding;

FIG. 4d is a fragmentary top view of the appliance, illustrating still further alternative shielding;

FIG. 5 is a side view of a human head including a brachytherapy appliance of the invention installed in an oral cavity of the head;

FIG. 5a is an enlarged fragmentary side view of the human head of FIG. 5, showing the appliance in relation to a cancer, and radiation being emitted toward the cancer;

FIG. 6 is a side view of aspects of another brachytherapy appliance according to the present invention, including a plurality of catheters for receiving radioactive tipped wires, respectively, for dosing a cancer with radiation;

FIG. 7 is a bottom view of the brachytherapy appliance of FIG. 6;

FIG. 8 is a sectional view taken along line 8-8 of FIG. 7;

FIG. 9 is another sectional view of the appliance of FIGS. 6 and 7, illustrated in association with teeth and a jaw in an oral cavity of a human head;

FIG. 10 is an illustration and a high level flow diagram, showing steps of a method of the invention for making a brachytherapy appliance;

FIG. 11 is a perspective view of a reservoir of liquid photopolymer, and a three-dimensional physical model of a body surface being produced by solidifying the liquid photopolymer using lasers;

FIG. 12 is a perspective view of a polymer block, including a three-dimensional model of a body surface to be milled from the block;

FIG. 13 is another perspective view of a polymer block of FIG. 12, illustrating a portion of the three-dimensional model milled therefrom;

FIG. 14 is a perspective view of a finished brachytherapy appliance that can be made using the solidified liquid photopolymer process of FIG. 11, or the milling process of FIGS. 12 and 13;

FIG. 14a is a perspective view of another finished brachytherapy appliance that can be made using the solidified liquid photopolymer process of FIG. 11, or the milling process of FIGS. 12 and 13;

FIG. 15 is sectional view taken through the appliance of FIG. 14a, illustrating one embodiment of channels or catheters that can be formed in the appliance as part of the lithography process of FIG. 11; and

FIG. 16 is another sectional view of the appliance of FIG. 14a, illustrating another embodiment of channels, that can be formed as part of the processes of either FIG. 11, or FIGS. 12 and 13.

### DETAILED DESCRIPTION OF PREFERRED EMBODIMENTS

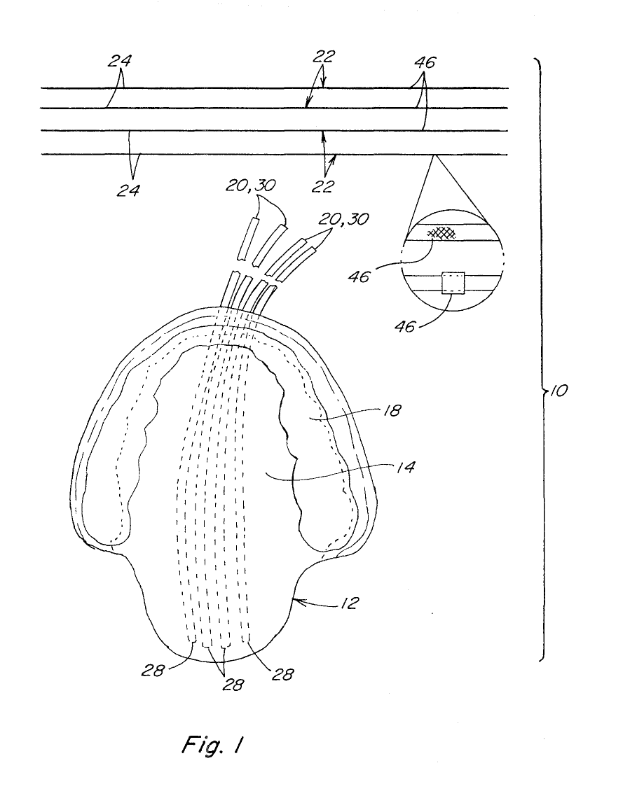

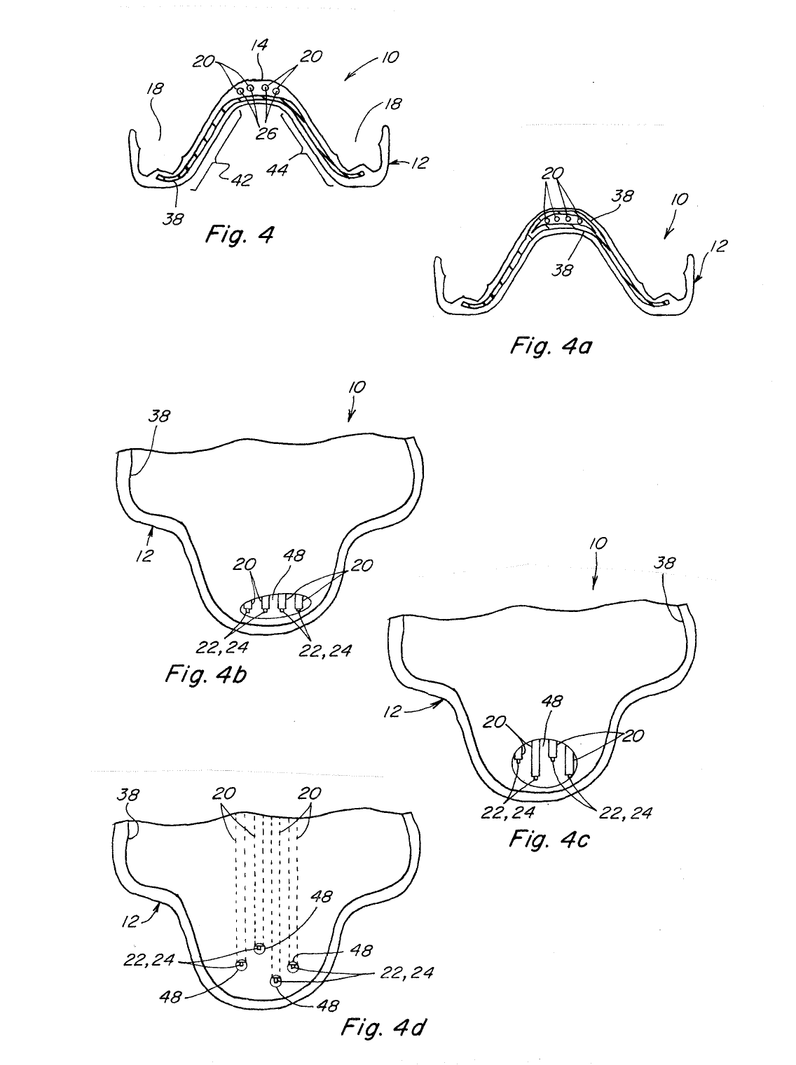

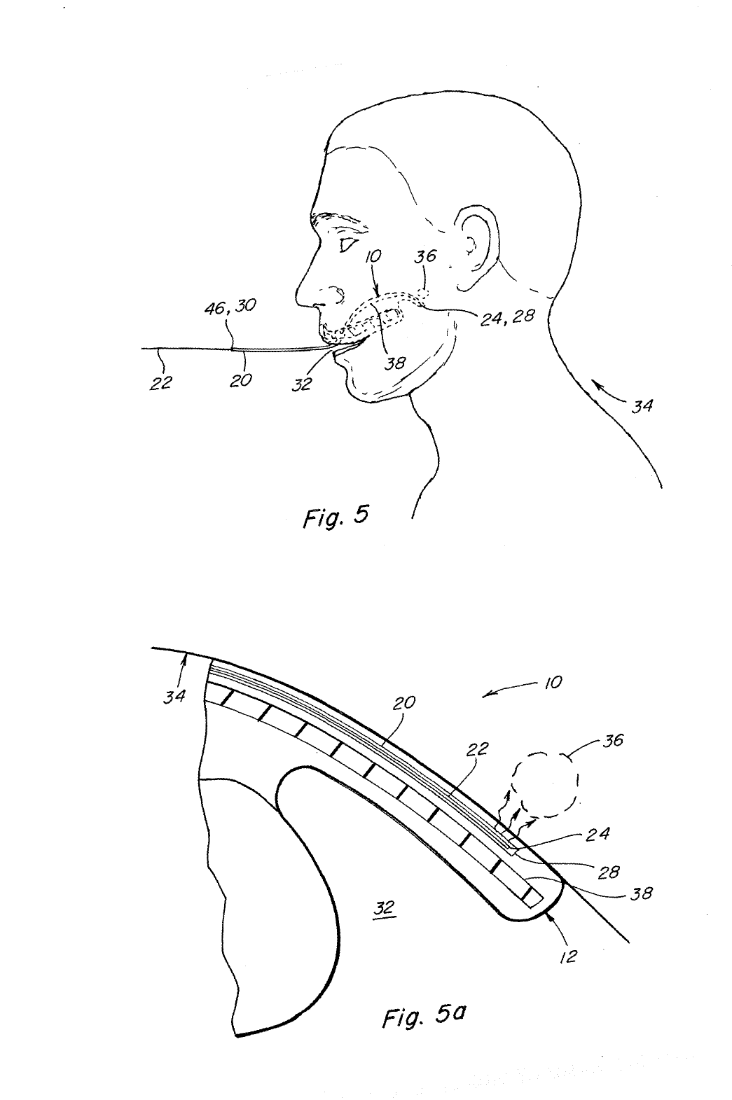

Referring now to FIGS. 1, 2, 3 and 4, wherein like numerals refer to like parts, a brachytherapy appliance 10 constructed and operable according to the teachings of the present invention, is shown. Brachytherapy appliance 10 is configured for use in cooperation with body surfaces within an oral cavity of a living body, which, as depicted hereinafter, is a human body. It should be recognized however, that the teachings of the invention have utility for brachytherapy appliances for use in cooperation with a wide variety of surfaces of a living body. Appliance 10 generally includes a body portion 12 which will be at least generally and preferably custom molded to the contours of a region of an oral cavity of a living body. More particularly, body portion 12 is molded to conform to the upper regions or palate of a human oral cavity, including a center region 14 molded to selected regions of the hard and/or the soft palate of the oral cavity, and a peripheral region 16 molded to the upper teeth, if any, and/or the endentulous ridge or arch, thereby forming a recess 18 extending partially around the center region 14, and contoured for cooperatively receiving and releasably attaching to the upper teeth, if any, or to the edentulous ridge or arch. Body portion 12 can be molded using common dental molding techniques and commercially available polymeric materials commonly used in the same, such as a well-known, commercially available dental acrylic material such as a methyl methacrylate. Importantly, at least one therapy delivery element is incorporated into appliance 10, which here preferably comprises at least one catheter 20 incorporated in or on body portion 12. Each catheter 20 is preferably adapted for cooperatively receiving and positioning radioactive media in desired relation to a cancer, which media here comprises an elongate wire 22 including a radiation emitting portion, which is preferably a radioactive tip 24 of iridium 192. Each catheter 20 is essentially a hollow tube defining a passage 26 (FIG. 4) having at least a terminal end portion 28 (FIG. 1) which is precisely positioned in relation to body portion 12, such that when a wire 12 is received in passage 26 with radioactive tip 24 located in the terminal end portion 28, tip 24 will be precisely positioned for irradiating a cancer or diseased tissue when appliance 10 is installed in the oral cavity.

The location, size and extent of the cancer will be determined by an oncology professional, and the radiological dosing will be determined by the oncology professional and/or physicist. From that information, the number and position of catheters 20 required will be determined by the professionals. Appliance 10 will be fabricated to include the required number of catheters 20, in the required position or positions, such that the therapy delivering region or regions of the wire or wires inserted into the catheter or catheters will be properly disposed for delivering the contemplated radiation dose to the cancer or diseased tissue. In this embodiment, this will entail the positioning of terminal end portions 28 of the catheters 20 in, or in connection with, molded body portion 12, such that the radioactive tips 24 of the wires when located in the catheters will be positioned for delivery of the required radiation dose to the cancer. Preferably, this will entail the fixed molding or affixing in place of catheters 20 within body portion 12, such that terminal end portions 28 will be fixed in location with respect to the cancer or diseased tissue when appliance 10 is installed in the oral cavity. Also preferably, this will entail allowing the extension of opposite ends 30 of catheters 20 from body portion 12, and thus also from the mouth 32 of a person 34 when appliance 10 is installed, as shown in FIG. 5. This will allow a healthcare professional to safely and conveniently insert wires 22 into catheters 20 with body portion 12 installed in the mouth, to position radioactive tips 24 at terminal end portions 28 within catheters 20, for delivering the required radiation dose to a cancer or diseased tissue, such as representative cancer 36, also illustrated in FIG. 5. At the end of the radiation therapy, wires 22 are removed from body portion 12, and body portion 12 removed from the mouth.

As noted hereinabove, radiation emitted by a therapeutic device, such as any of radioactive tips 24 of wires 22, can potentially unintentionally injure or damage noncancerous body parts, tissue, glands and/or organs, such as the teeth, salivary glands, olfactory nerves, taste buds, and the like. To avoid such injury or damage, appliance 10 includes one or more shields, such as a radiation shield 38 as variously illustrated in FIGS. 4, 4a, 4b, 4c and 4d, in association with body portion 12, positioned or disposed for providing shielding of desired tissue regions, glands, organs, and/or other body parts, from radiation emitted by tips 24. A shield, such as radiation shield 38, can comprise a suitable radiation absorbing or containing material, such as lead. To protect non-diseased body portions, such as the tissue of the superior region of the oral cavity, such as the endentulous arch, the tongue, teeth, and the like, located in the vicinity of a shield, such as shield 38, from unhealthy effects from exposure to the material of the shield, the shield is preferably encapsulated by a layer of protective material, for instance, by one or more layers of the polymeric material constituting body portion 12, such as an acrylic or the like. A shield can be encapsulated within body portion 12 when molded, or can be installed on a surface thereof, then coated with the protective layer. Here, for instance, terminal and portions 28 of catheters 20 are disposed in the posterior region of body portion 12, for irradiating a cancer in the posterior region of the oral cavity, or the upper throat region, as generally illustrated by cancer 36 in FIGS. 5 and 5a. Shield 38 for this location, can cover the inferior or lower or lateral surface of body portion 12 which will face the oral cavity, generally denoted as region 40 in FIG. 3, to protect the healthy tissue, as well as those portions of the upper teeth and gums that face and therefore could be injured by subjection to radiation emitted by tips 24 when installed in catheters 20 (FIG. 5a), as generally denoted by regions 42 and 44 in FIG. 4.

Here, it should be noted that it is contemplated that the shielding to be used in association with a brachytherapy appliance of the invention, such as appliance 10, can take any of a variety of forms, as desired or required for providing protection to tissue and other body parts from radiation emitted by the radioactive elements of the appliance. For instance, the shielding can take the form of an envelope (FIGS. 4a, 4b, 4c and 4d) covering both the superior and/or inferior surfaces of catheters 20, as well as, anterior and/or posterior shielding and lateral shielding. More particularly in this regard, it is contemplated that an appliance according to the invention could include catheters 20 which are substantially completely ensconced within radiation shielding, with the exception of one or more precisely placed and oriented windows, such as a window or windows 48 (FIGS. 4b, 4c and 4d) for the emission of radiation toward a cancer or diseased tissue.

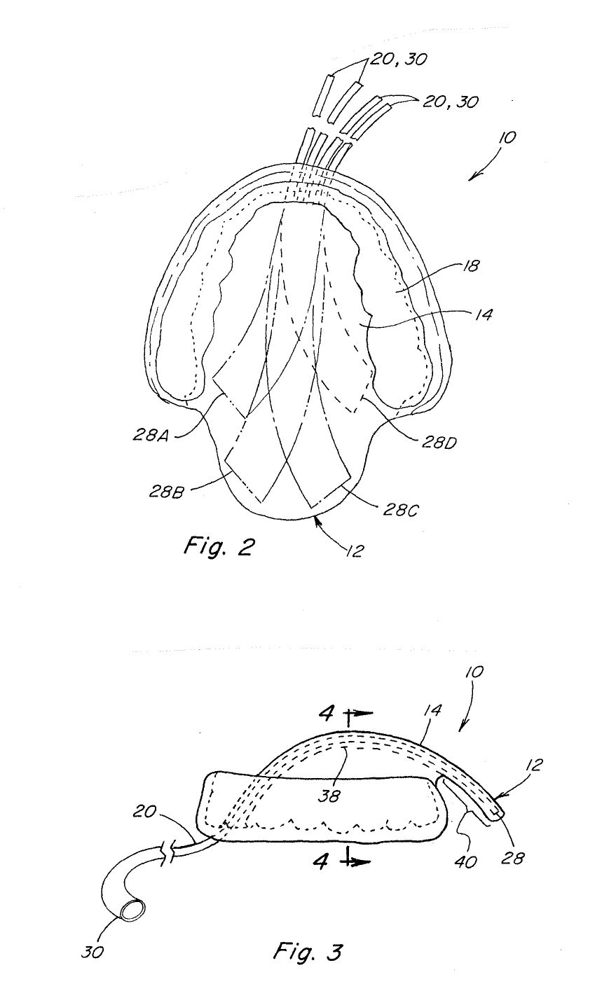

Referring more particularly to FIG. 2, as noted above, it is also contemplated that catheters 20, and more particularly, the terminal end portions 28 or other portions thereof which are to hold the radiation emitting elements, can be positioned at any location on or in connection with an appliance of the invention, such as appliance 10, for facilitating delivery of the radiation dose required for treatment of a cancer. This is denoted by numerals 28A, 28B, 28C and 28D, which illustrate alternative locations for terminal and portions 28, both in the anterior/posterior and lateral directions.

Still further, it is contemplated according to the invention that catheters 20 can be of the same or a different length, for positioning radiation emitting portions of wires, such as tips 24 of wires 22, in desired relation to a cancer for dosing the cancer with radiation, as illustrated in FIGS. 4b, 4c and 4d. For instance, terminal end portions 28 or other radiation positioning portions of a plurality of catheters 20 could be arranged in a generally circular, oval, offset, or other shaped array deemed most effective for dosing a cancer with radiation, as illustrated in FIGS. 4c and 4d. A plurality of catheters 20 can also be arranged in a three-dimensional array, again, such as deemed most effective for dosing a cancer with radiation.

As another feature of the invention, wires 22 and/or catheters 20 can include indicia for indicating when wires 22 are fully received within catheters 20, respectively, for positioning the radiation emitting portions of the wires, such as radioactive tips 24, at the desired position in relation to a cancer. For instance, such indicia can include a mark or markings along the length of a wire 22, such as a dyed or etched spot, notch, or the like, as generally denoted by numerals 46 in FIG. 1, which will match up with an end 30 of a catheter 20 (FIG. 5), such that when the mark or marking is coincident with, or in other predetermined relation with, end 30, a healthcare professional inserting the wire 22 into a catheter 20 will know that the wire is properly and fully positioned within the catheter. The properly positioned wire or wires 22 will then be left in the catheter or catheters 20 for the prescribed time, and then removed when the radiation dose has been delivered. Alternatively, or additionally, a wire or wires 22 could include a hard stop, such as a sharp bend or crease at the location therealong to be coincident with the end 30 of conduit 20, to provide the desired indication of proper positioning.

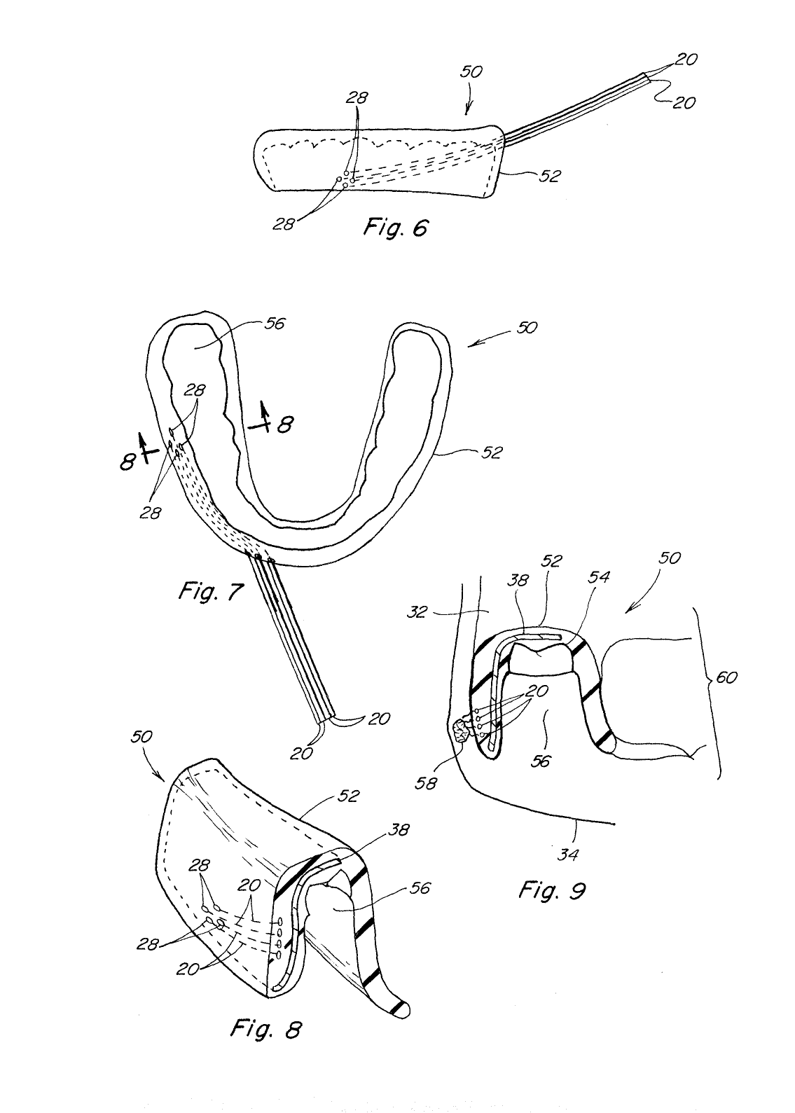

Referring also to FIGS. 6, 7, 8 and 9, another brachytherapy appliance 50 constructed and operable according to the teachings of the present invention, is shown, like parts of appliance 50 and appliance 10 being identified by like numerals. Brachytherapy appliance 50, like appliance 10, is configured for use in an oral cavity of a living body, which, as depicted in FIG. 9, is a human body. Appliance 50 generally includes a body portion 52 which will be at least generally and preferably custom molded to the contours of a region of an oral cavity of a living body. More particularly, body portion 52 is molded to conform to the lower teeth 54, if any, and/or the lower endentulous ridge or arch, thereby forming a recess 56 contoured for cooperatively receiving and releasably attaching to the lower teeth 54, if any, or to the lower edentulous ridge or arch. Body portion 52, like body portion 12, can be molded using common dental molding techniques and commercially available polymeric materials commonly used in the same, such as a well-known, commercially available dental acrylic material such as a methyl methacrylate. Importantly, at least one therapy delivery element is incorporated into appliance 50, which here preferably comprises at least one catheter 20 incorporated in or on body portion 52. Each catheter 20 is preferably adapted for cooperatively receiving and positioning radioactive media in desired relation to a cancer, which media here comprises an elongate wire 22 (illustrated in other Figures) including a radiation emitting portion, which is preferably a radioactive tip of iridium 192, as explained above. Each catheter 20 is essentially a hollow tube defining a passage having at least a terminal end portion 28 which is precisely positioned in relation to body portion 52, such that when a wire 12 is receivable in the passage with radioactive tip 24 located in the terminal end portion 28, tip 24 will be precisely positioned for irradiating a cancer or diseased tissue when appliance 50 is installed in the oral cavity.

The location, size and extent of the cancer will be determined by an oncology professional, and the radiological dosing will be determined by the oncology professional and/or physicist. From that information, the number and position of catheters 20 required will be determined by the professionals. Appliance 50 will be fabricated to include the required number of catheters 20, in the required position or positions, such that the therapy delivering region or regions of the wire or wires inserted into the catheter or catheters will be properly disposed for delivering the contemplated radiation dose to the cancer or diseased tissue. In this embodiment, this will entail the positioning of terminal end portions 28 of the catheters 20 in, or in connection with, molded body portion 52, such that the radioactive tips 24 of the wires when located in the catheters will be positioned for delivery of the required radiation dose to the cancer. Preferably, this will entail the fixed molding in place of catheters 20 within body portion 52, such that terminal end portions 28 will be fixed in location with respect to the cancer or diseased tissue when appliance 50 is installed in the oral cavity. Also preferably, this will entail allowing the extension of opposite ends 30 of catheters 20 from body portion 52, and thus also from the mouth 32 of a person 34 when appliance 50 is installed, as shown in FIG. 9. This will allow a healthcare professional to safely and conveniently insert wires 22 into catheters 20 with body portion 52 installed in the mouth, to position radioactive tips 24 at terminal end portions 28 within catheters 20, for delivering the required radiation dose to a cancer or diseased tissue, such as representative cancer 58, also illustrated in FIG. 9. At the end of the radiation therapy, wires 22 are removed from body portion 52, and body portion 52 removed from the mouth.

As noted hereinabove in relation to appliance 10, radiation emitted by a therapeutic device, such as any of radioactive tips 24 of wires 22, can potentially unintentionally injure or damage noncancerous body parts, tissue, glands and/or organs, such as the teeth, salivary glands, olfactory nerves, taste buds, and the like. To avoid such injury or damage, appliance 50 includes one or more shields, such as a radiation shield 38 as illustrated in FIGS. 8 and 9, in association with body portion 52, positioned or disposed for providing shielding of desired tissue regions, glands, organs, and/or other body parts, from radiation emitted by tips 24. A shield, such as radiation shield 38, can comprise a suitable radiation absorbing or containing material, such as lead. To protect non-diseased body portions, such as the tissue of the inferior and superior regions of the oral cavity, such as the endentulous arch, the tongue, teeth, and the like, located in the vicinity of a shield, such as shield 38, from unhealthy effects from exposure to the material of the shield, the shield is preferably encapsulated by a layer of protective material, for instance, by one or more layers of the polymeric material constituting body portion 52, such as an acrylic or the like. A shield can be encapsulated within body portion 52 when molded, or can be installed on a surface thereof, then coated with the protective layer. Here, for instance, terminal and portions 28 of catheters 20 are disposed in the lateral region of body portion 52, for irradiating a cancer in the cheek region of the oral cavity, as generally illustrated by cancer 58 in FIG. 9. Shield 38 for this location, can cover the region of body portion 52 which will face the healthy tissue to be shielded, generally denoted as region 60 in FIG. 9, to protect the healthy tissue, as well as those portions of the teeth and gums that face and therefore could be injured by subjection to radiation emitted by tips 24 when installed in catheters 20.

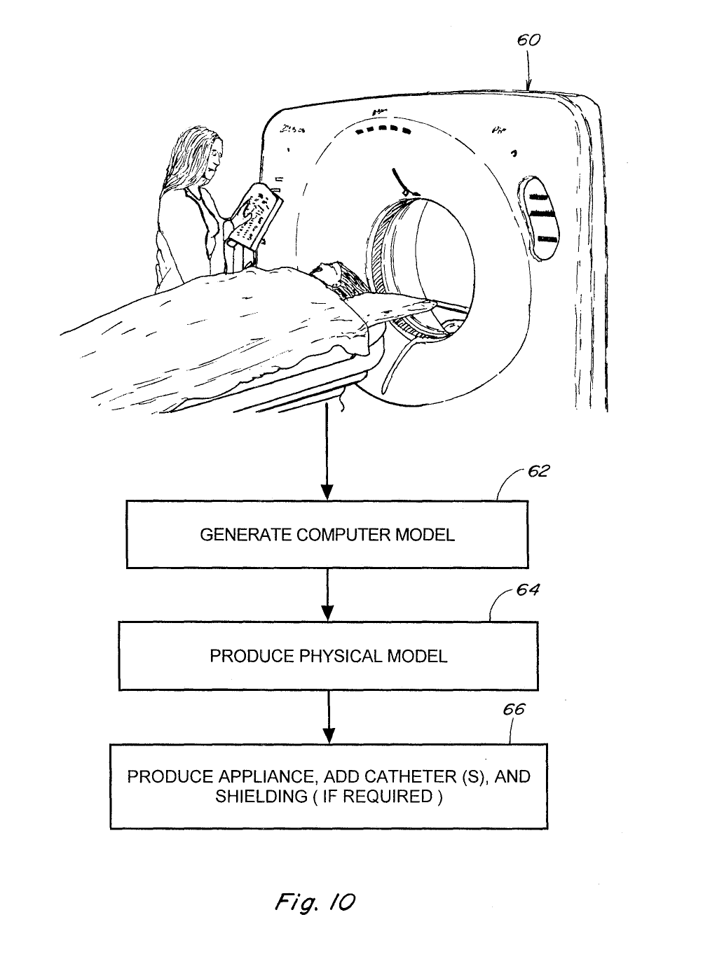

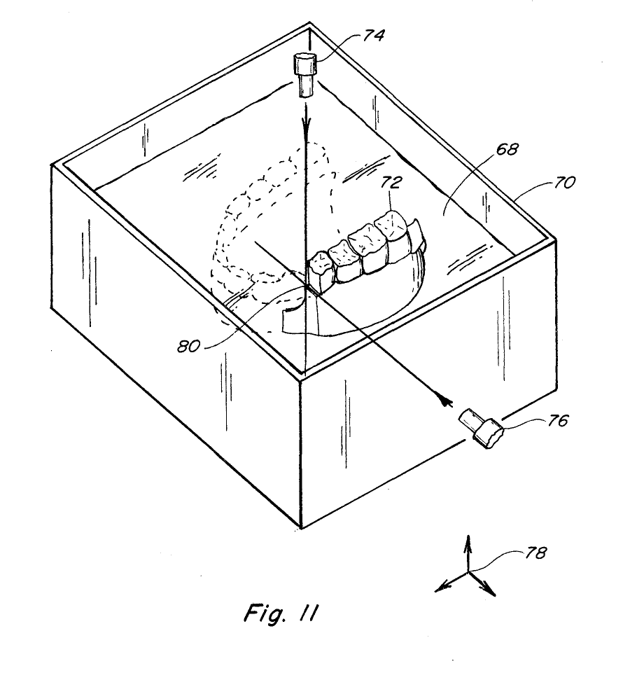

Referring also to FIGS. 10, 11, 12 and 13, steps of additional methods according to the invention for producing a brachytherapy appliance are illustrated, and in FIGS. 14, 15 and 16, a brachytherapy appliance illustrative of those that can be made by any of such additional methods, is shown. Essentially, in the additional methods of the invention, a computerized model or map of the topography of a surface of a portion of a body against which a brachytherapy appliance is to be positioned, is developed. Referring in particular to FIG. 10, using a suitable scanning device capable of modeling or mapping a body surface, such as, but not limited to, a tomography device such as a CT scanning device, e.g., a conventional multi-slice CT (MDCT) scanner, a dental (cone-beam) CT scanner, or an MRI scanner, as represented by scanning device 60, the computer model can be generated, as denoted at step 62. X-ray data and/or data from a laser scan of the oral cavity can also or alternatively be used for developing the computerized data set. Manual interpretation and surface feature discrimination of data can also be used as required, for achieving the desired accuracy. The objective of the map is to provide a shape to which the appliance will be modeled, having sufficient detail for the purposes of the present invention, which is to position a therapeutic element or elements, e.g., radioactive tipped wire or wires, in desired position and proximity to diseased tissue, for delivering a therapeutic treatment to the tissue.

It is contemplated and anticipated that, as an advantage of the invention, in many instances, at least one of scan of the pertinent body region will be or have been performed as a matter of course in a diagnosis of the disease and/or determination of the location and/or extent of the diseased tissue, or for other more routine dental related purposes. It is also contemplated that such a diagnostic scan or scans will include imaging data which encompasses the surface of the body portion against which the brachytherapy appliance is sought is to be placed, which data will thus have utility for generating the surface model or map for the present purposes. Using an existing scan is advantageous for a number of reasons, including that eliminates need for a new scan and thus limits radiation exposure, which can be an important factor for patients about to receive radiation therapy, and it can eliminate a need for physically molding the body surface itself, e.g., the making of impressions. This can be particularly desirable when the patient is sickly, recovering from a surgery, and/or unable to open his or her mouth to the extent required for making an impression of the subject surface with conventional dental molding materials. This is also desirable in the event the subject surface is in a location wherein the molding operation may generate a gag reflex and/or discomfort or pain, such as more toward the rear of the oral cavity. If an existing scan is not available, one can be performed, with the advantage that it will not be as invasive as making a dental impression.

As still another advantage, the computerized surface map can be generated at any time subsequent to the scan or scans, and additional physical access to the patient is not required. Such mapping will include either or both soft (e.g., soft tissue) and hard (e.g., tooth) surfaces, depending on the required positioning of the appliance.

From the map, a three dimensional physical model of the body surface will be produced, as denoted at step 64. This three dimensional physical model can be of the body surface against which, or adjacent to which, the appliance is to be placed, and thus to which the appliance will be molded or conformed, or it can be the surface of the appliance itself that will be placed against, or adjacent to, the body surface, that is, so as to essentially be a mirror image or mating surface to the subject body surface. The appliance is then produced, using the model, as denoted by step 66. Here, if the three dimensional model is the mirror or mating surface to the body surface, the model will form all or a portion of the body of the appliance.

If the physical model is of the body surface, the appliance can be made in any suitable manner, by molding or conforming the appliance to the model, including in any of the ways described above for making appliances 10 and 50, for instance, by molding, thermal or vacuum forming or otherwise conforming the appliance to the physical model, e.g., wherein the catheter or catheters are positioned on the model and are held in place using wax or adhesives, and set a required or desired distance from the surface of the physical model. Shielding can be applied or incorporated as required or directed by the physician or physicist, and a shell, such as a thermal form or vacuum form plastic shell, can be formed or placed on the model and catheter or catheters, and the shielding, if present, to hold them in place. The formed plastic shell is then trimmed if required and the appliance is complete. As an alternative, the catheter or catheters, or representations thereof can be located on the physical model, and the appliance molded or formed thereover, for incorporating the catheter or catheters into the appliance.

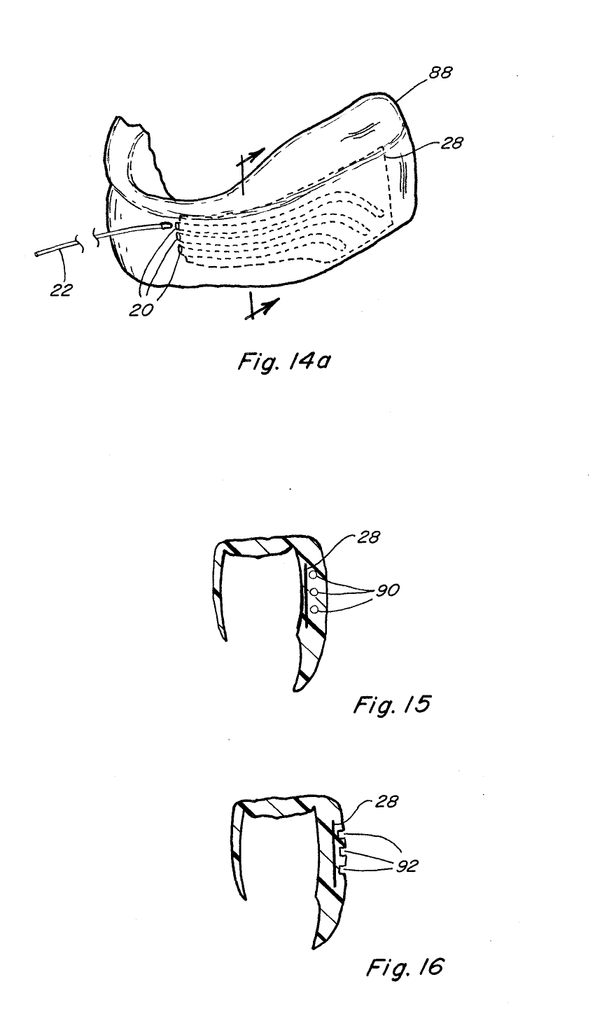

If the three-dimensional physical model is of the appliance side of the surface, the model can be formed so as to comprise essentially the complete appliance, or just the portion or layer thereof which will be placed against, or adjacent to, the subject body surface. In either instance, the computerized map can be adapted to also include locations for an element or elements to serve as, or to receive, the catheter or catheters, for instance, one or more grooves and/or fully or partially enclosed channels, positioned and routed as determined by the oncology professional or physicist. This is illustrated in FIGS. 15 and 16 discussed below. Additional features, such as the surface shape and contours of the opposite side of the appliance can be incorporated into the model.

Referring more particularly to FIG. 11, the physical model can be produced, for instance, using a commercially available computer controlled lithography process, such as a stereo lithography process wherein a liquid photopolymer 68 contained in a reservoir 70 is selectively cured into a three dimensional model 72, using beams emitted from appropriate light or radiation sources 74 and 76 based on the map data. The light or radiation sources can comprise, for instance, but are not limited to, lasers or ultraviolet lights. Essentially, this process relies on precisely controlled relative movements of the polymer reservoir and light or radiation beams, as denoted by arrows 78, for the selective curing of the polymer at the intersection 80 of the beams, as controlled by a computer based on the three dimensional map data.

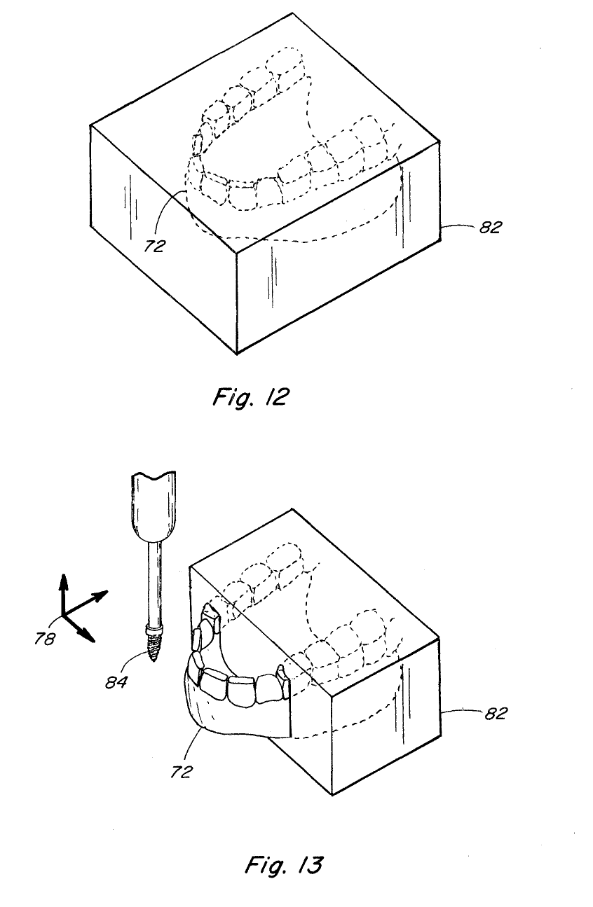

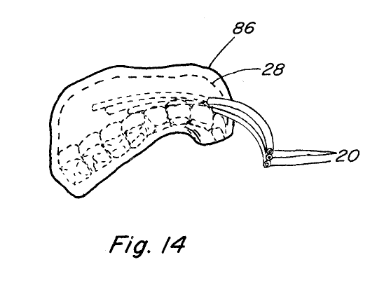

Referring more particularly to FIGS. 12 and 13, the physical model 72 can also be produced by computer controlled milling machine, from a urethane or other suitable polymer block 82. As in the stereo lithography process, the computer will control the milling operation based on the three dimensional map data to produce the replica of the body surface, the milling cutter 84 and block 82 being relatively precisely movable, as denoted by arrows 76, for forming model 72. Again, this surface can be the body surface itself, as illustrated in FIG. 13, or the mirror image or mating surface that forms part of the appliance. In the latter instance, the dotted lines of model 72 illustrated in FIG. 12 will represent the features and contours of the inner surface of the appliance itself, that will be positioned against or adjacent to the subject body surfaces. Here, it should be noted that milling cutter 84 is intended to be representative of a variety of cutters that can be used, but is not intended to be limiting in configuration, shape and/or size.

FIGS. 14 and 14a illustrate additional appliances 86 and 88 that can be made using the processes of FIGS. 11, 12 and 13, like parts of appliances 86 and 88 and 10 and 52, being identified by like numerals. Appliance 86 is configured for placement on a lower jaw, and appliance 88 being configured for placement on an upper jaw, each including multiple catheters 20 for receiving wires, as illustrated by wire 22, in the above-discussed manner, and shields 28. FIGS. 15 and 16 are sectional views of appliance 88, including passages 90 and grooves 92, respectively, that can either form the catheters 20 (passages 90), or receive them (passages 90 and grooves 92), and which can be computer positioned as part of the mapping process and/or the manufacturing process, and incorporated into the appliance when made. Appliance 88 in these views also includes appropriately positioned shields 28.

Thus, there has been shown and described a novel brachytherapy appliance and method, which overcomes many of the problems and shortcomings set forth above. It will be apparent, however, to those familiar in the art, that many changes, variations, modifications, and other uses and applications for the subject device are possible. All such changes, variations, modifications, and other uses and applications that do not depart from the spirit and scope of the invention are deemed to be covered by the invention which is limited only by the claims which follow.

What is claimed is:

1. A brachytherapy appliance, comprising:
a body portion;
at least one catheter disposed on the body portion, the catheter being configured for receiving at least one element having a radiation emitting portion; and
at least one element having a radiation emitting portion cooperatively receivable in the at least one catheter;
wherein the body portion has a contoured shape computer modeled to a contoured shape of a surface of a living body so as to conform at least substantially thereto, so as to be positionable in at least substantially conforming relation to the surface, and wherein the at least one catheter is positioned such that the radiation emitting portion when received therein will be positioned by the catheter in a predetermined position in relation to the body portion.

2. The appliance of claim 1, further comprising a radiation shield incorporated into at least one portion of the body portion.

3. The appliance of claim 1, wherein the at least one catheter is fixedly formed in the body portion.

4. The appliance of claim 1, wherein the element comprises a wire and the radiation emitting portion comprises a tip of the wire.

5. The appliance of claim 1, comprising a plurality of the catheters and a plurality of the elements including the radiation emitting portions cooperatively receivable in the catheters, respectively.

6. The appliance of claim 5, wherein the catheters are arranged in a predetermined array within the body portion.

7. The appliance of claim 1, wherein the at least one catheter comprises a tube.

8. The appliance of claim 1, wherein the contoured shape of the body portion has a contoured shape of a surface within an oral cavity of a living body.

9. The appliance of claim 8, wherein the contoured shape of the body portion has a shape of an endentulous arch within an oral cavity.

10. A method of making a brachytherapy appliance, comprising steps of:
generating a computerized three dimensional model of a surface of a living body, from at least one computerized scan of the living body;
producing a three dimensional physical model of the surface of the living body from the computerized three dimensional model; and
producing a brachytherapy appliance from the three dimensional physical model, the appliance including a surface conforming at least substantially to the surface of the living body and so as to be positionable thereon in at least substantially conforming relation thereto, the appliance including at least one conduit configured for receiving a therapy delivery element, the conduit being disposed in a predetermined position on the appliance so as to be located in predetermined relation to the surface of the living body when the appliance is positioned in the conforming relation thereto.

11. The method of claim 10, wherein the step of producing the brachytherapy appliance from the three dimensional physical model comprises molding or conforming the surface of the appliance to the model, such that the surface of the appliance will be positionable in the at least substantially conforming relation to the surface of the living body.

12. The method of claim 10, wherein the three dimensional physical model comprises a mirror image of the surface of the living body, and the step of producing the brachytherapy appliance from the three dimensional physical model comprises incorporating the physical model into the appliance.

13. The method of claim 10, wherein at least a portion of at least one of the three dimensional physical model and the appliance is produced by a computer controlled lithography process.

14. The method of claim 13, wherein the three dimensional physical model is produced using the computer controlled lithography process.

15. The method of claim 13, wherein the at least one conduit is produced in the appliance using the computer controlled lithography process.

16. The method of claim 10, wherein at least a portion of one of the three dimensional physical model and the appliance is produced by a computer controlled milling process.

17. The method of claim 16, wherein the three dimensional physical model is produced using the computer controlled milling process.

18. The method of claim 16, wherein the at least one conduit is formed in the appliance using the computer controlled milling process.

19. The method of claim 10, wherein the therapy delivery element comprises a radiation emitting element.

20. The method of claim 19, wherein the radiation emitting element comprises a tip of a wire.

21. The method of claim 19, wherein the step of producing the brachytherapy appliance comprises placing a radiation shield in predetermined relation to the at least one conduit.

22. The method of claim 19, wherein the at least one conduit comprises a catheter, and the radiation emitting element comprises a wire insertable into the catheter.
